(19) United States
(12) Patent Application Publication
(10) Pub. No.: US 2014/0171445 A1
(43) Pub. Date: Jun. 19, 2014

(54) TREATMENT AND PREVENTION OF DISEASES MEDIATED BY MICROORGANISMS VIA DRUG-MEDIATED MANIPULATION OF THE EICOSANOID BALANCE

(71) Applicants: Katrin Mayer-Barber, North Bethesda, MD (US); Bruno de Bezerril Andrade, Rockville, MD (US); Alan Sher, Potomac, MD (US); Daniel Leo Barber, North Bethesda, MD (US)

(72) Inventors: Katrin Mayer-Barber, North Bethesda, MD (US); Bruno de Bezerril Andrade, Rockville, MD (US); Alan Sher, Potomac, MD (US); Daniel Leo Barber, North Bethesda, MD (US)

(73) Assignee: The USA, as represented by the Secretary, Department of Health and Human Services, Bethesda, MD (US)

(21) Appl. No.: 14/236,940

(22) PCT Filed: Aug. 2, 2012
(86) PCT No.: PCT/US2012/049280

§ 371 Date: Feb. 4, 2014

Related U.S. Application Data
(60) Provisional application No. 61/515,237, filed on Aug. 4, 2011.
(60) Provisional application No. 61/515,229, filed on Aug. 4, 2011.

(51) Int. Cl. A61K 31/381; A61K 45/06; A61K 31/41; A61K 31/47; A61K 31/404; A61K 31/5575; A61K 31/145

(52) U.S. Cl. CPC A61K 31/381; A61K 31/5575; A61K 45/06; A61K 31/145; A61K 31/47; A61K 31/404; A61K 31/41
USPC 514/254.11; 514/443; 514/354; 514/255.06; 514/311; 514/415; 514/382

(57) ABSTRACT

The invention provides a method of enhancing the efficacy of antibiotic treatment of tuberculosis, trypanosomiasis, leprosy, and leishmaniasis involving co-administering to a mammal undergoing antibiotic treatment therapeutically effective amounts of a first compound that is an inhibitor of 5-lipoxygenase and optionally a second compound that is a product of the cyclooxygenase pathways. The invention also provides a pharmaceutical composition comprising an antibiotic, an inhibitor of 5-lipoxygenase, and a product of the cyclooxygenase pathways.

### CROSS-REFERENCE TO RELATED APPLICATIONS

This patent application claims the benefit of U.S. Provisional Patent Application No. 61/515,229, filed Aug. 4, 2011, and U.S. Provisional Patent Application No. 61/515,237, filed Aug. 4, 2011, which are incorporated by reference.

### BACKGROUND OF THE INVENTION

Diseases such as tuberculosis, trypanosomiasis, leprosy, and leishmaniasis are known to be caused by microorganisms. These diseases cause death and disfigurement of the afflicted. For example, tuberculosis remains a leading cause of death. There are approximately 8 million active cases of tuberculosis per year, with 3 million deaths annually ascribed thereto. About 1.7 billion people are estimated to harbor the latent Mycobacterium tuberculosis infection.

Currently, the treatment of tuberculosis consists of administering a combination of four first line drugs, isoniazid, rifampicin, ethambutol, and pyrazinamide, administered individually as a single drug formulation or as a fixed dose combination. For effective treatment the aforementioned four first line drugs are given to a patient in the initial or induction phase, during which the drugs are used in combination to kill the rapidly multiplying population of M. tuberculosis as well as to prevent the emergence of drug resistance. This is followed by a continuation phase during which sterilizing drugs, isoniazid, rifampicin, and pyrazinamide are given to kill the intermittently dividing population of M. tuberculosis.

Currently, such diseases require long-term treatment with antibiotics. Interruption of treatment or use of inadequate dosage strengths can lead to recurrence of diseases and to development of drug resistance in patients. There remains a need for improved therapy of such diseases.

### BRIEF SUMMARY OF THE INVENTION

The invention provides a method of treating or preventing a disease mediated or caused by intracellular microorganisms comprising administering to a mammal therapeutically effective amounts of at least one compound that is an inhibitor of the 5-lipoxygenase pathway, wherein the disease is selected from the group consisting of tuberculosis, trypanosomiasis, leprosy, and leishmaniasis.

The invention also provides a pharmaceutical composition comprising effective amounts of (a) an inhibitor of the 5-lipoxygenase pathway and (b) a product of the cyclooxygenase pathways, and optionally (c) an antimicrobial agent.

The invention additionally provides a kit for enhancing the effective immune response of a mammal in the treatment of a disease caused by intracellular microorganisms, wherein the kit comprises effective amounts of (a) an inhibitor of the 5-lipoxygenase pathway and (b) a product of the cyclooxygenase pathways.

The invention further provides a method of treating or preventing a disease caused by intracellular microorganisms comprising administering effective amounts of (a) an antimicrobial agent, (b) an inhibitor of the 5-lipoxygenase pathway, and (c) a product of the cyclooxygenase pathways.

### BRIEF DESCRIPTION OF THE SEVERAL VIEWS OF THE DRAWING(S)

FIG. 1 illustrates the arachidonic acid cascade.

FIG. 2 illustrates the change in weight over time in C57BL6 mice infected with Mycobacterium tuberculosis treated with poly-ICLC with and without further treatment with zileuton and PGE2.

FIG. 3 illustrates the survival over time in C57BL6 mice infected with Mycobacterium tuberculosis treated with poly-ICLC with and without further treatment with zileuton and PGE2.

FIG. 4 illustrates the survival over time in IL-1a/bDKO−/− (IL-1α/β double knock-out) mice infected with Mycobacterium tuberculosis with and without further treatment with zileuton and PGE2.

FIG. 5 illustrates the effect on the number of colony forming units in the lungs of C57BL6 mice infected with Mycobacterium tuberculosis treated with poly-ICLC alone, with poly-ICLC and PGE2, with poly-ICLC, PGE2, and zileuton, and with poly-ICLC and zileuton.

FIG. 6 illustrates the survival over time in IL-1a/bDKO−/− (IL-1α/β double knock-out) mice infected with Mycobacterium tuberculosis with and without further treatment with dapsone and PGE2.

### DETAILED DESCRIPTION OF THE INVENTION

The invention provides a method of treating or preventing a disease mediated or caused by intracellular microorganisms comprising administering to a mammal therapeutically effective amounts of a first compound that is an inhibitor of the 5-lipoxygenase pathway, wherein the disease is selected from the group consisting of tuberculosis, trypanosomiasis, leprosy, and leishmaniasis.

In certain embodiments, the inhibitor of the 5-lipoxygenase pathway is an inhibitor of 5-lipoxygenase.

In certain of the above embodiments, the inhibitor of 5-lipoxygenase is a compound of the formula:

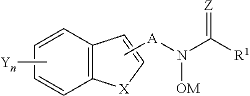

wherein R¹ is hydrogen, C₁ to C₄ alkyl, C₂ to C₄ alkenyl, or NR²R³ wherein R² and R³ are independently selected from hydrogen, C₁ to C₄ alkyl and hydroxyl, but R² and R³ are not simultaneously hydroxyl;

wherein X is oxygen, sulfur, SO₂, or NR⁴, wherein R⁴ is hydrogen, C₁ to C₆ alkyl, C₁ to C₆ alkoyl, aroyl, or alkylsulfonyl;

A is selected from C₁ to C₆ alkylene and C₂ to C₆ alkenylene, each of which is linear or branched;

n is 1-5;

Y is independently selected from the group consisting of hydrogen, halogen, hydroxy, cyano, halosubstituted alkyl, C₁-C₁₂ alkyl, C₂-C₁₂ alkenyl, C₁-C₁₂ alkoxy, C₃-C₈ cycloalkyl, C₁-C₈ thioalkyl, aryl, aryloxy, aroyl, C₆-C₁₀ aryl-C₁-C₁₂ alkyl, C₆-C₁₀ aryl-C₂-C₁₁ alkenyl, C₆-C₁₀ aryl-C₁-C₁₂ alkoxy, C₆-C₁₀ arylthio-C₁-C₁₂ alkoxy, and substituted derivatives of aryl, aryloxy, aroyl, C₆-C₁₀ aryl-C₁-C₁₂ alkyl, C₆-C₁₀ aryl-C₂-C₁₂ alkenyl, C₆-C₁₀ aryl-C₁-C₁₂ alkoxy, or C₆-C₁₀ arylthio-C₁-C₁₂ alkoxy, wherein the substituents are selected from halo, nitro, cyano, C₁-C₁₂ alkyl, alkoxy, and halosubstituted alkyl;

Z is oxygen or sulfur; and

M is hydrogen, a pharmaceutically acceptable cation, aroyl, or C₁ to C₁₂ alkanoyl,

r a pharmaceutically acceptable salt thereof or stereoisomer thereof,

or a compound selected from the group consisting of:

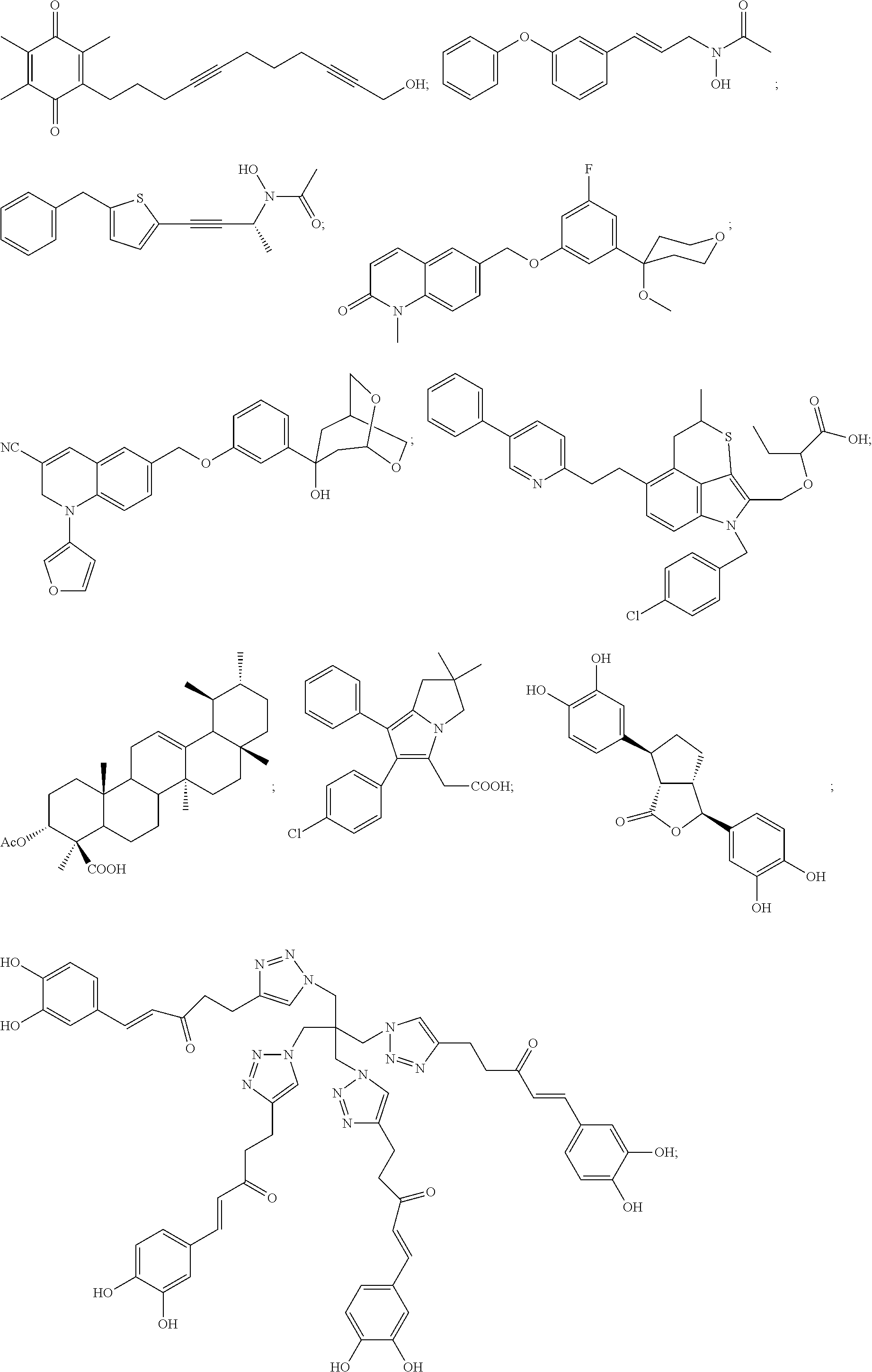

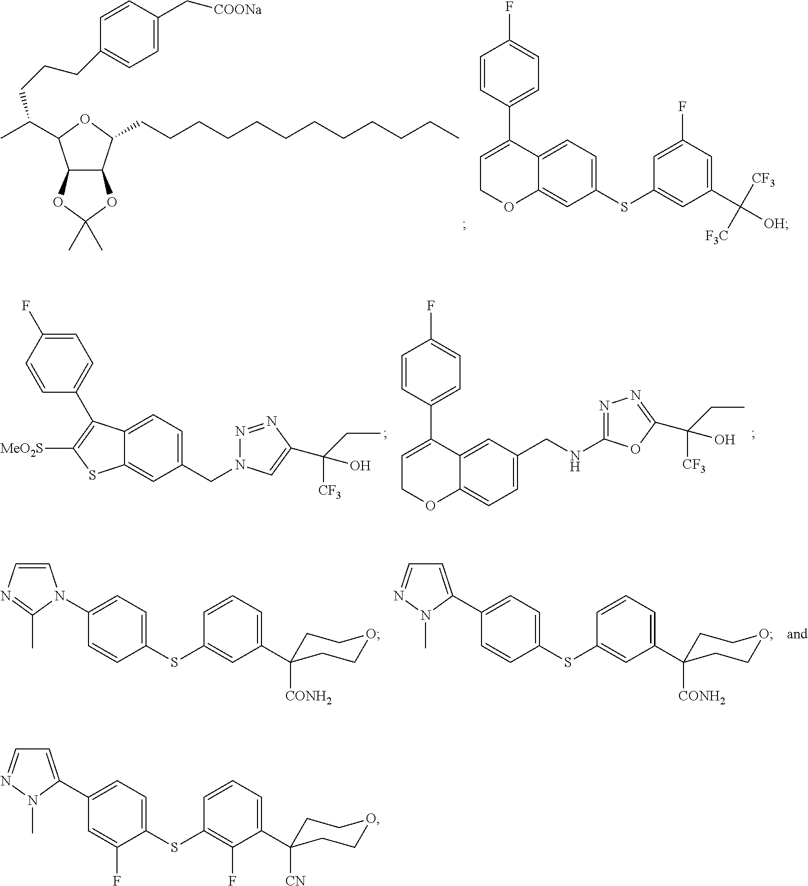

or a pharmaceutically acceptable salt thereof or stereoisomer thereof.

In a preferred embodiment, the inhibitor of 5-lipoxygenase is zileuton, which has the formula:

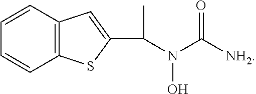 [SMILES: CC(c1cc2ccccc2s1)N(O)C(N)=O]

Zileuton is a marketed drug from Abbott Laboratories (Abbott Park, Ill.). The other inhibitors of 5-lipoxygenase are described in C. Pergola et al., Expert Opin. Ther. Pat. 2010, March, 20(3), 355-375.

In another preferred embodiment, the inhibitor of 5-lipoxygenase is dapsone, which is 4,4′-diaminodiphenylsulfone.

In certain embodiments, the inhibitor of the 5-lipoxygenase pathway is a leukotriene receptor antagonist or a lipoxin receptor antagonist. The receptor antagonists can be any suitable receptor antagonist. For example, the leukotriene receptor antagonist can be selected from the group consisting of montelukast, zafirlukast, and pranlukast.

When any of the aforesaid inhibitors of the 5-lipoxygenase pathway contains one or more basic or acidic moieties that can exist as a salt (e.g., a basic nitrogen atom, a carboxylic acid, or a hydroxamic acid), the inhibitor of 5-lipoxygenase can be administered in the form of the parent compound or can be administered in the form of a pharmaceutically acceptable salt. The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington 's Pharmaceutical Sciences, 18th ed., Mack Publishing Company, Easton, Pa., 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety (e.g., a carboxylic acid or a hydroxamic acid) include sodium and potassium salts. Preferred pharmaceutically acceptable salts of inventive compounds having a basic moiety (e.g., a tertiary amine or a basic nitrogen-containing heterocyclic ring) include hydrochloride and hydrobromide salts. The compounds of the present invention containing an acidic or basic moiety are useful in the form of the free base or acid or in the form of a pharmaceutically acceptable salt thereof.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

With respect to the aforesaid inhibitors of the 5-lipoxygenase pathway, when the compound or salt has a single asymmetric carbon atom, the compound or salt may exist as a racemate, i.e., as mixtures of equal amounts of optical isomers, i.e., equal amounts of two enantiomers. The compound or salt of Formula (I) or (II) may exist in the form of a single enantiomer. As used herein, “single enantiomer” is intended to mean a compound that comprises more than 50% of a single enantiomer. When the compound or salt has more than one chiral center, and can therefore exist as a mixture of diastereomers, the compound or salt can exist as a mixture of diastereomers or can exist in the form of a single diastereomer, or as a mixture wherein a distereomer is in excess over another disastereomer, e.g., more than 50% of a single diastereomer.

In certain embodiments, the method further comprises administering at least one product of the cyclooxygenase pathways to the mammal. The cyclooxygenase can be COX-1 (i.e., PGH synthase-1) or COX-2 (i.e., PGH synthase-2). In a preferred embodiment, the COX-2 dependent prostaglandin is prostaglandin E2 (i.e., PGE2). In another embodiment, the COX-2 dependent prostaglandin is prostaglandin F2 (e.g., PGF2 and/or PGF2α). When the cyclooxygenase is COX-2, the product of the cyclooxygenase pathways can be described as a COX-2 dependent prostaglandin.

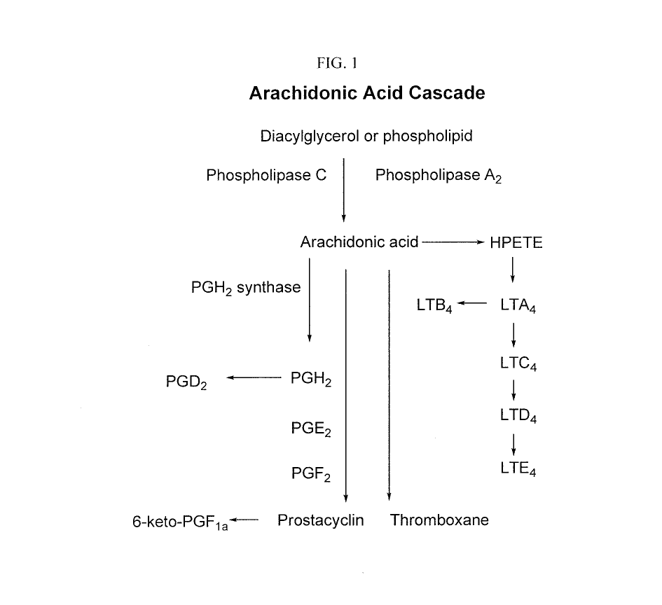

The term “eicosanoid” refers to any of the class of compounds derived from polyunsaturated fatty acids, such as arachidonic acid and linolinic acid, and involved in cellular activity. Eicosanoids result from oxidation of arachidonic acid via the arachidonic acid cascade, which is illustrated in FIG. 1.

The term “oxygenase” refers to any of the class of enzymes that catalyze the incorporation of molecular oxygen into its substrate.

The term “enhancing” the biological activity, function, health, or condition of an mammal refers to the process of augmenting, fortifying, strengthening, or improving.

“Preventing” within the context of the present invention, refers to a prophylactic treatment of an individual prone or subject to development of a condition, in particular, a disease mediated or caused by intracellular microorganisms, for example, wherein the disease is selected from the group consisting of tuberculosis, trypanosomiasis, leprosy, and leishmaniasis. For example, those of skill in the medical arts may be able to determine, based on clinical symptoms and patient history, a statistical predisposition of a particular individual to the development of a disease mediated or caused by intracellular microorganisms. For example, a history of exposure to a disease mediated or caused by intracellular microorganisms can be used to assess the predisposition of a particular individual to the development of the disease and thus inform the individual as to the desirability of preventative treatment with an inhibitor of the 5-lipoxygenase pathway and COX-2 dependent prostaglandin, salts thereof or stereoisomers thereof, or a medicament formed therefrom. Accordingly, an individual predisposed to the development of a disease mediated or caused by intracellular microorganism, such as a disease selected from the group consisting of tuberculosis, trypanosomiasis, leprosy, and leishmaniasis, may be treated with an inhibitor of the 5-lipoxygenase pathway and COX-2 dependent prostaglandin, salts thereof or stereoisomers thereof in order to prevent, inhibit, or slow the development of the disease.

In certain embodiments, the inhibitor of the 5-lipoxygenase pathway can be administered to the mammal using any suitable method. For example, the inhibitor of the 5-lipoxygenase pathway can be administered in the form of a pharmaceutical composition(s) comprising a pharmaceutically acceptable carrier and an inhibitor of the 5-lipoxygenase pathway.

In certain embodiments, the inhibitor of the 5-lipoxygenase pathway and COX-2 dependent prostaglandin can be administered to the mammal using any suitable method. For example, the inhibitor of the 5-lipoxygenase pathway and/or COX-2 dependent prostaglandin can be administered in the form of a pharmaceutical composition(s) comprising a pharmaceutically acceptable carrier and an inhibitor of the 5-lipoxygenase pathway and/or COX-2 dependent prostaglandin. In some embodiments, the inhibitor of the 5-lipoxygenase pathway and COX-2 dependent prostaglandin can be administered in separate pharmaceutical compositions. In other embodiments, the inhibitor of the 5-lipoxygenase pathway and COX-2 dependent prostaglandin can be administered in a single pharmaceutical composition.

It is preferred that the pharmaceutically acceptable carrier be one that is chemically inert to the active compounds and one that has no detrimental side effects or toxicity under the conditions of use.

The choice of carrier will be determined in part by the particular compound of the present invention chosen, as well as by the particular method used to administer the composition. Accordingly, there is a wide variety of suitable formulations of the pharmaceutical composition of the present invention. The following formulations for oral, aerosol, nasal (e.g, intranasal), pulmonary, parenteral, subcutaneous, intravenous, intraarterial, intramuscular, intraperitoneal, intrathecal, intratumoral, topical, rectal, and vaginal administration are merely exemplary and are in no way limiting.

The pharmaceutical composition can be administered parenterally, e.g., intravenously, intraarterially, subcutaneously, intradermally, or intramuscularly. Thus, the invention provides compositions for parenteral administration that comprise a solution or suspension of the inventive compound or salt dissolved or suspended in an acceptable carrier suitable for parenteral administration, including aqueous and non-aqueous isotonic sterile injection solutions.

Overall, the requirements for effective pharmaceutical carriers for parenteral compositions are well known to those of ordinary skill in the art. See, e.g., Banker and Chalmers, eds., Pharmaceutics and Pharmacy Practice, J. B. Lippincott Company, Philadelphia, pp. 238-250 (1982), and Toissel, ASHP Handbook on Injectable Drugs, 4th ed., pp. 622-630 (1986). Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound or salt of the present invention may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations can contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations, including those that are useful for transdennal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Formulations suitable for oral administration can consist of (a) liquid solutions, such as a therapeutically effective amount of the inventive compound dissolved in diluents, such as water, saline, or orange juice, (b) capsules, sachets, tablets, lozenges, and troches, each containing a predetermined amount of the active ingredient, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compound or salt of the present invention, alone or in combination with other suitable components, can be made into aerosol formulations to be administered via inhalation. The compounds are preferably supplied in finely divided form along with a surfactant and propellant. Typical percentages of active compound are 0.01%-20% by weight, preferably 1%-10%. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25%-5%. The balance of the composition is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the compound or salt of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

It will be appreciated by one of ordinary skill in the art that, in addition to the aforedescribed pharmaceutical compositions, the compound or salt of the present invention may be formulated as inclusion complexes, such as cyclodextrin inclusion complexes, or liposomes. Liposomes serve to target the compounds to a particular tissue, such as lymphoid tissue or cancerous hepatic cells. Liposomes can also be used to increase the half-life of the inventive compound. Liposomes useful in the present invention include emulsions, foams, micelles, insoluble monolayers, liquid crystals, phospholipid dispersions, lamellar layers and the like. In these preparations, the active agent to be delivered is incorporated as part of a liposome, alone or in conjunction with a suitable chemotherapeutic agent. Thus, liposomes filled with a desired inventive compound or salt thereof, can be directed to the site of a specific tissue type, hepatic cells, for example, where the liposomes then deliver the selected compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream. A variety of methods are available for preparing liposomes, as described in, for example, Szoka et al., Ann. Rev. Biophys. Bioeng., 9, 467 (1980), and U.S. Pat. Nos. 4,235,871, 4,501,728, 4,837,028, and 5,019,369. For targeting to the cells of a particular tissue type, a ligand to be incorporated into the liposome can include, for example, antibodies or fragments thereof specific for cell surface determinants of the targeted tissue type. A liposome suspension containing a compound or salt of the present invention may be administered intravenously, locally, topically, etc. in a dose that varies according to the mode of administration, the agent being delivered, and the stage of disease being treated.

In certain embodiments, the method further comprises administering at least one antimicrobial agent to the mammal. Suitable antimicrobial agents include antibiotic agents, atiprotozoal agents, and combinations thereof.

The inventive method desirably enhances the efficacy of antimicrobial treatment of a disease caused by intracellular microorganisms comprising co-administering to a mammal undergoing antibiotic treatment for a disease selected from the group consisting of tuberculosis, trypanosomiasis, leprosy, and leishmaniasis. The antibiotic can be any one or more antibiotics suitable for treatment of the aforesaid diseases.

When the disease is tuberculosis, the antimicrobial is typically an antibiotic selected from the group consisting of isoniazid, rifampin, pyrazinamide, ethambutol, and combinations thereof. The combination of the aforesaid antibiotics is well known in the medical arts as suitable first line therapy for tuberculosis. The dosage of isoniazid, rifampin, pyrazinamide, ethambutol can be as typically used for the treatment of tuberculosis.

In a preferred embodiment, the disease is tuberculosis caused by infection with one or more members of the Mycobacterium tuberculosis complex (MTC). The Mycobacterium tuberculosis consists of Mycobacterium africanum, Mycobacterium bovis, Mycobacterium canettii, Mycobacterium kansasii, Mycobacterium microti, and Mycobacterium tuberculosis. In a more preferred embodiment, the disease is tuberculosis caused by infection with Mycobacterium tuberculosis.

In an embodiment, the tuberculosis is a multi-drug resistant tuberculosis (MDR). Multi-drug resistant tuberculosis is defined as TB that is resistant at least to isoniazid and rifampicin. MDR tuberculosis develops during treatment of fully sensitive TB when the course of antibiotics is interrupted and the levels of drug in the body are insufficient to kill 100% of bacteria.

In an embodiment, the tuberculosis is an extremely drug resistant tuberculosis (XRT). Extremely drug resistant tuberculosis can develop when patients having tuberculosis are given anti-tuberculosis drugs but at insufficient doses or at improper intervals.

In certain embodiments, the disease is selected from the group consisting of trypanosomiasis, leprosy, and leishmaniasis. In these embodiments, the antimicrobial agent is typically an antiprotozoal agent selected from the group consisting of melarsoprol, nifurtimox, pentamidine, sodium stibuglyconate, suramin, atovapuone, timidazole, dapsone, clofazinime, and rifampin, and combinations thereof.

The proposed immunotherapeutic strategy documented in this invention has potential application for the treatment of a number of infections in addition to Mycobacterium tuberculosis. In particular leprosy, Chagas' Disease (American trypanosomiasis) and leishmaniasis are three global infectious diseases that in common with Mtb are caused by intracellular pathogens. Studies in experimental animal models have implicated arachidonic acid metabolites in the regulation of host resistance to these infections and the pathways involved are potential targets to for the treatment strategy described herein (Reiner et al., J. Immunology, 1985, January 134(1): 556-63; Machado et al., Adv. Parasitol. 2011, 76:1-31; Fink et al. J. Leukoc. Biol. 2010, March; 87(3):361-3).

Desirably, administration of a 5-lipoxygenase pathway inhibitor and optionally a product of the cyclooxygenase pathways enhance the efficacy of antibiotic treatment by enhancing the immune response of the mammal being treated. Preferably, the enhancing results in reducing overall disease severity and mortality, reducing the length of antibiotic treatment regimen, increased tolerance of antibiotic, or any combination thereof. Inhibition of the 5-lipoxygenase pathway and treatment with a product of the cyclooxygenase pathways results in alteration of the eicosanoid balance in a mammal treated therewith. It is believed that the synergistic effects of altering the eicosanoid balance and treatment with antibiotics results in a more efficient reduction in bacterial burden and immunopathology, thereby reducing overall disease severity and mortality. As a result, it is believed that the required period for antibiotic administration can be shortened and the antibiotic dosage lowered which can lead to reduced toxicity (and thereby increased tolerance of antibiotic) and lowered incidence of drug resistance. In addition, because of targeted effects on the innate immune response, the inventive method may have particular advantages in the treatment of tuberculosis in T cell deficient HIV patients.

The antibiotic, inhibitor of the 5-lipoxygenase pathway, and/or the product of the cyclooxygenase pathways can be administered simultaneously, sequentially or cyclically. For example, the antibiotic, inhibitor of the 5-lipoxygenase pathway, and the product of the cyclooxygenase pathways can be administered in a single pharmaceutical composition. In another embodiment, for example, the antibiotic can be administered in separate pharmaceutical compositions, e.g., within a short period of time. In other embodiments, the antibiotic can be administered for a period of time. Subsequently, the inhibitor of the 5-lipoxygenase pathway, and the product of the cyclooxygenase pathways can be administered together, with or without the co-administration of the antibiotic. In some embodiments, administration of the antibiotic, inhibitor of the 5-lipoxygenase pathway, and the product of the cyclooxygenase pathways ccan be alternated. Additional embodiments will be readily understood by one of ordinary skill in the medical arts.

The dose administered to a mammal in accordance with the present invention should be sufficient to effect the desired response. Such responses include reversal or prevention of the bad effects of the disease for which treatment is desired or to elicit the desired benefit. One skilled in the art will recognize that dosage will depend upon a variety of factors, including the age, condition, and body weight of the mammal, as well as the source, particular type of the disease, and extent of the disease in the mammal. The size of the dose will also be determined by the route, timing and frequency of administration as well as the existence, nature, and extent of any adverse side-effects that might accompany the administration of a particular compound and the desired physiological effect. It will be appreciated by one of skill in the art that treatment of tuberculosis may require prolonged treatment involving multiple administrations.

Suitable doses and dosage regimens of the inhibitor of 5-lipoxygenase and COX-2 dependent prostaglandin can be determined by conventional range-finding techniques known to those of ordinary skill in the art. Generally, treatment is initiated with smaller dosages that are less than the optimum doses of the inhibitor of 5-lipoxygenase and COX-2 dependent prostaglandin. Thereafter, the dosage is increased by small increments until the optimum effect under the circumstances is reached. The present inventive method typically will involve the administration of about 0.1 to about 300 mg of one or more of the inhibitor of 5-lipoxygenase and about 0.1 to about 300 μg of the COX-2 dependent prostaglandin per kg body weight of the mammal.

By way of example and not intending to limit the invention, the dose of the inhibitor of 5-lipoxygenase for methods of treating tuberculosis can be about 0.001 to about 1 mg/kg body weight of the subject being treated per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. The dose of the COX-2 dependent prostaglandin for methods of treating tuberculosis can be about 0.001 to about 1 μg/kg body weight of the subject being treated per day, for example, about 0.001 μg, 0.002 μg, 0.005 μg, 0.010 μg, 0.015 μg, 0.020 μg, 0.025 μg, 0.050 μg, 0.075 μg, 0.1 μg, 0.15 μg, 0.2 μg, 0.25 μg, 0.5 μg, 0.75 μg, or 1 μg/kg body weight per day.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the inventive methods can provide any amount of any level of treatment, prevention, amelioration, or inhibition of the disorder in a mammal. For example, a disorder, including symptoms or conditions thereof, may be reduced by, for example, 100%, 90%, 80%, 70%, 60%, 50%, 40%, 30%, 20%, or 10%. Furthermore, the treatment, prevention, amelioration, or inhibition provided by the inventive method can include treatment, prevention, amelioration, or inhibition of one or more conditions or symptoms of the disorder, e.g., cancer. Also, for purposes herein, “treatment,” “prevention,” “amelioration,” or “inhibition” can encompass delaying the onset of the disorder, or a symptom or condition thereof.

The term “mammal” includes, but is not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simioids (monkeys) or of the order Anthropoids (humans and apes). An especially preferred mammal is the human. Furthermore, the subject can be the unborn offspring of any of the forgoing hosts, especially mammals (e.g., humans), in which case any screening of the subject or cells of the subject, or administration of compounds to the subject or cells of the subject, can be performed in utero.

The invention also provides a pharmaceutical composition comprising effective amounts of (a) an inhibitor of the 5-lipoxygenase pathway, and/or (b) product of the cyclooxygenase pathways, and (c) optionally an antimicrobial.

The invention further provides a kit for enhancing the effective immune response of a mammal in the treatment or prevention of tuberculosis, wherein the kit comprises effective amounts of (a) an inhibitor of the 5-lipoxygenase pathway and (b) product of the cyclooxygenase pathways, and instructions to treat or prevent a disease caused by intracellular microorganisms.

The invention additionally provides a method for treating or preventing a disease caused by intracellular microorganisms. The method comprises administering to the mammal effective amounts of (a) an antimicrobial agent and (b) an inhibitor of the 5-lipoxygenase pathway, and optionally (c) a product of the cyclooxygenase pathways.

The following examples further illustrate the invention but, of course, should not be construed as in any way limiting its scope.

C57BL/6 mice were purchased from Taconic Farms, Inc. (Gennantown, N.Y.). IL-1a/bDKO−/− mice are maintained at the National Institutes of Health.

PGE2 was purchased from Sigma-Aldrich Corp. (St. Louis, Mo.). Zileuton was obtained as Zyflo (Abbott Laboratories, North Chicago, Ill.).

### Example 1

This example demonstrates the effect of co-administration of zilueton and PGE2 to C57BL6 mice infected with Mycobacterium tuberculosis that are concurrently treated with poly-ICLC.

Four groups of five C57BL/6 mice (“B6 mice”) were used in this study. All four groups were exposed to M. tuberculosis at a level of 100-150 colony forming units via intranasal aerosol route. A control group of five mice was not further treated. A comparative group was treated twice weekly via intranasal administration of poly-ICLC, which is polyinosinic-polycytidylic acid condensed with poly-L-lysine and carboxymethylcellulose (Oncovir Inc., Washingon, D.C.). The comparative group of five mice was not further treated. A test group of five mice was treated with zileuton, which was administered in drinking water at a concentration of 6 mg/mL, PGE2, which was administered intranasally at a concentration of 6 μg/30 μLin phosphate buffered saline per mouse twice a week, and poly-ICLC. A second control group of five mice was treated with zileuton and PGE2, but was not treated with poly-ICLC.

Intranasal poly-ICLC has been shown to exacerbate tuberculosis in mice through the pulmonary recruitment of a pathogen-permissive monocyte/macrophage population. Antonelli, L. R. V., et al., J. Clin. Investigation 2010, 120(3), 1674-1682.

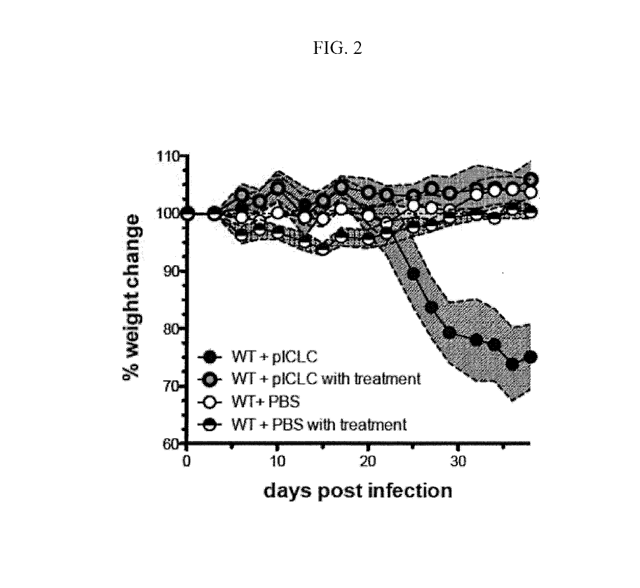

The mean value of the weight of the surviving mice in each of the three groups was followed over time. After 31 days, the control group of B6 mice had a mean weight that was approximately 103% of their starting weight. The second control group of B6 mice, which was treated with zileuton and PGE2, but was not with poly-ICLC, had a mean weight that was approximately 100% of their starting weight. The test group which was treated with zileuton and PGE2 and with poly-ICLC, had a mean weight that was approximately 105% of their starting weight. The comparative group which was treated with poly-ICLC alone had a mean weight that was approximately 74% of their starting weight. The results are depicted graphically in FIG. 2.

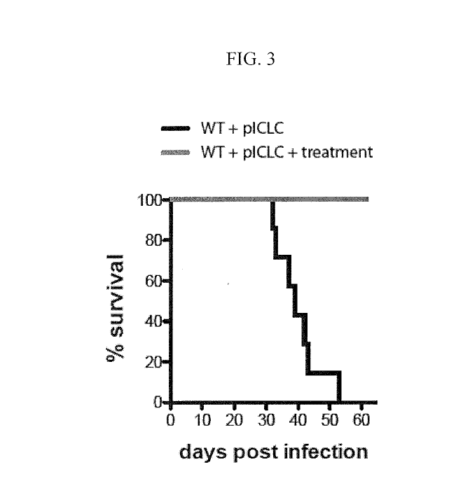

In addition, 100% of the test group which was treated with zileuton and PGE2 and with poly-ICLC survived past day 53 post-infection. None of the comparative group which was treated with poly-ICLC alone survived past day 53 post-infection. Survival over time for the comparative group, which was treated with poly-ICLC, and for the test group, which was treated with zileuton and PGE2 and with poly-ICLC, is depicted graphically in FIG. 3.

Thus, treatment of poly-ICLC treated tuberculosis-infected mice which are further treated with zileuton and PGE2 results in survival and weight retention as compared to poly-ICLC treated tuberculosis-infected mice which are not further treated.

### Example 2

Two groups of five IL-1a/bDKO−/− (IL-1α/β double knock-out) mice and one group of five C57BL/6 mice were used in this study. The C57BL/6 mice were used as a control.

All three groups were exposed to M. tuberculosis at a level of 100-150 colony forming units via intranasal aerosol route. A test group of five IL-1a/bDKO−/− mice and a control group of C57BL/6 mice were treated with zileuton, which was administered in drinking water at a concentration of 6 mg/mL, and PGE2, which was administered intranasally at a concentration of 6 g/30 μLin phosphate buffered saline per mouse twice a week. A comparative group of IL-1a/bDKO−/− mice was not treated with zileuton and PGE2.

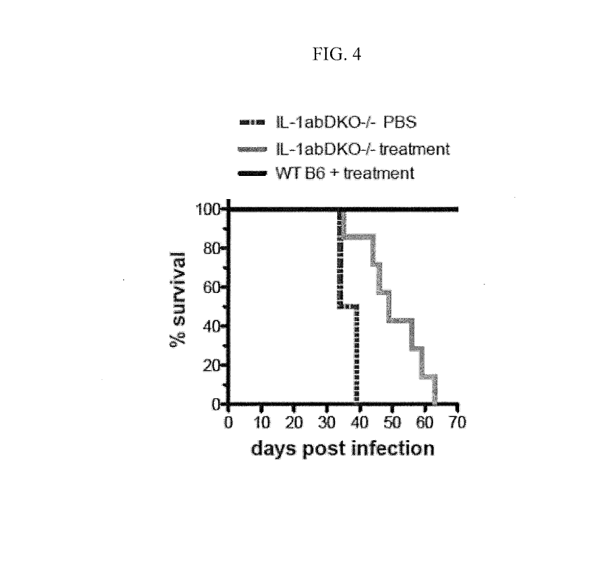

None of the comparative group of IL-1a/bDKO−/− mice survived past 40 days post-infection. One of the test group of five IL-1a/bDKO−/− mice died at day 40, with the remaining four mice surviving more than 40 days but less than about 65 days. All of the control group of C57BL/6 mice survived more than 60 days. The survival over time for the three groups is depicted in FIG. 4.

It is known that the cytokine IL-1 is central in inducing protective prostaglandins and mice that lack IL-1 die of experimental tuberculosis infection. See, for example, Mayer-Barber et al., J. Immunol. 2010 184:3326-3330; published ahead of print Mar. 3, 2010, doi:10.4049/jimmunol.0904189.

The results of this example demonstrate that by altering the eicosanoid balance in M. tuberculosis infected IL-1a/bDKO−/− mice by treatment with zileuton and PGE2 enhances survival of the mice.

### Example 3

C57BL6 mice were infected with 200 CFU of Mtb by the aerosol route and given poly-ICLC twice a week. One group of mice were treated with PBS as a control and was not treated with poly-ICLC. A second group of mice was not further treated. A third group of mice was further treated with PGE2. A fourth group of mice was further treated with PGE2 and zileuton. A fifth group of mice was further treated with zileuton alone.

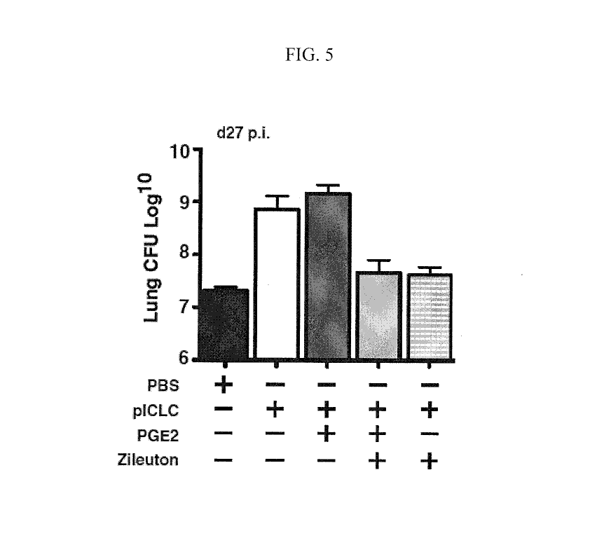

After a period of time, the colony forming units (“CFU”) in lungs were determined for each group of mice, and the results graphically illustrated in FIG. 5.

As is apparent from the results depicted in FIG. 5, the control group had approximately 7.4 log¹⁰ CFU. Mtb-infected poly-ICLC-treated mice had approximately 8.9 log¹⁰ CFU. Mtb-infected poly-ICLC-treated mice that were further treated with PGE2 had approximately 9.2 log¹⁰ CFU. Mtb-infected poly-ICLC-treated mice that were further treated with PGE2 had approximately 8.9 log¹⁰ CFU. Mtb-infected poly-ICLC-treated mice that were further treated with PGE2 and with zileuton had approximately 7.6 log¹⁰ CFU. Mtb-infected poly-ICLC-treated mice that were further treated with zileuton alone had approximately 7.6 log¹⁰ CFU.

### Example 4

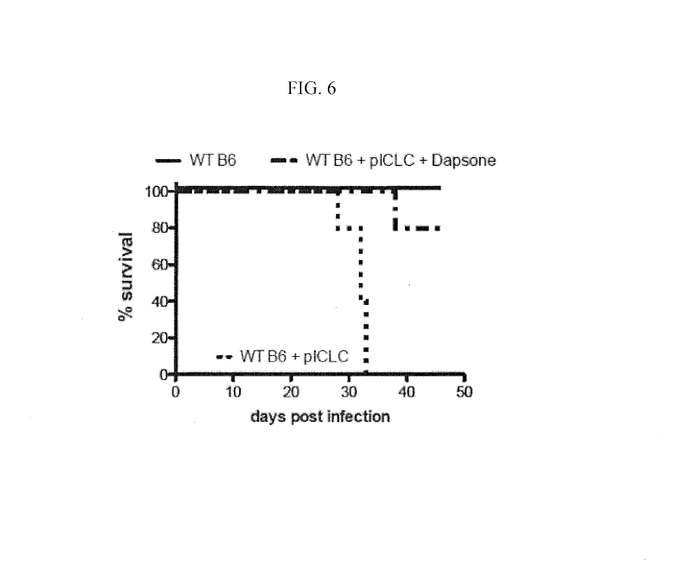

The experiment described in Example 2 was repeated, except that dapsone was substituted for zileuton. The survival over time for the three groups of mice is depicted in FIG. 6.

The results of this example demonstrate that by altering the eicosanoid balance in M. tuberculosis infected IL-1a/bDKO−/− mice by treatment with dapsone and PGE2 enhances survival of the mice.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing a disease caused by intracellular microorganisms comprising administering to a mammal therapeutically effective amounts of at least one compound that is an inhibitor of the 5-lipoxygenase pathway, wherein the disease is tuberculosis.

2. The method of claim 1, wherein the inhibitor of the 5-lipoxygenase pathway is an inhibitor of 5-lipoxygenase.

3. The method of claim 2, wherein the inhibitor of 5-lipoxygenase is a compound of the formula: 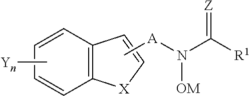
wherein R¹ is hydrogen, C₁ to C₄ alkyl, C₂ to C₄ alkenyl, or NR²R³ wherein R² and R³ are independently selected from hydrogen, C₁ to C₄ alkyl and hydroxyl, but R² and R³ are not simultaneously hydroxyl;
wherein X is oxygen, sulfur, SO₂, or NR⁴, wherein R⁴ is hydrogen, C₁ to C₆ alkyl, C₁ to C₆ alkanoyl, aroyl, or alkylsulfonyl;
A is selected from C₁ to C₆ alkylene and C₂ to C₆ alkenylene, each of which may be linear or branched;
n is 1-4;
Y is independently selected from the group consisting of hydrogen, halogen, hydroxy, cyano, halosubstituted alkyl, C₁-C₁₂ alkyl, C₂-C₁₂ alkenyl, C₁-C₁₂ alkoxy, C₃-C₈ cycloalkyl, C₁-C₈ thioalkyl, aryl, aryloxy, aroyl, C₆-C₁₀ aryl-C₁-C₁₂ alkyl, C₆-C₁₀ aryl-C₂-C₁₁ alkenyl, C₆-C₁₀ aryl-C₁-C₁₂ alkoxy, C₆-C₁₀ arylthio-C₁-C₁₂ alkoxy, and substituted derivatives of aryl, aryloxy, aroyl, C₆-C₁₀ aryl-C₁-C₁₂ alkyl, C₆-C₁₀ aryl-C₂-C₁₂ alkenyl, C₆-C₁₀ aryl-C₁-C₁₂ alkoxy, or C₆-C₁₀ arylthio-C₁-C₁₂ alkoxy, wherein the substituents are selected from halo, nitro, cyano, C₁-C₁₂ alkyl, alkoxy, and halosubstituted alkyl;
Z is oxygen or sulfur; and
M is hydrogen, a pharmaceutically acceptable cation, aroyl, or C₁ to C₁₂ alkanoyl,
or a pharmaceutically acceptable salt thereof or stereoisomer thereof;
or a compound selected from the group consisting of: 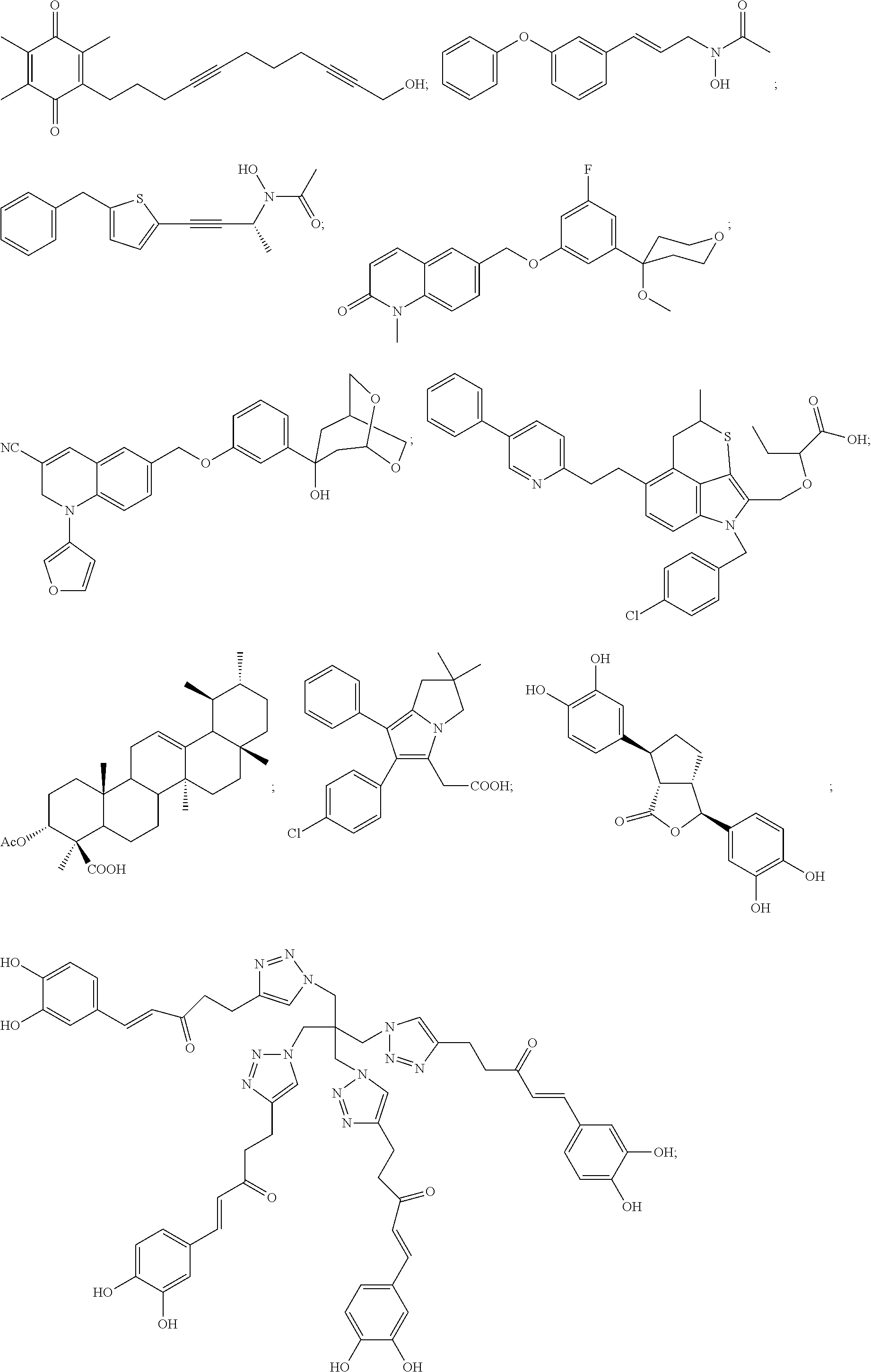 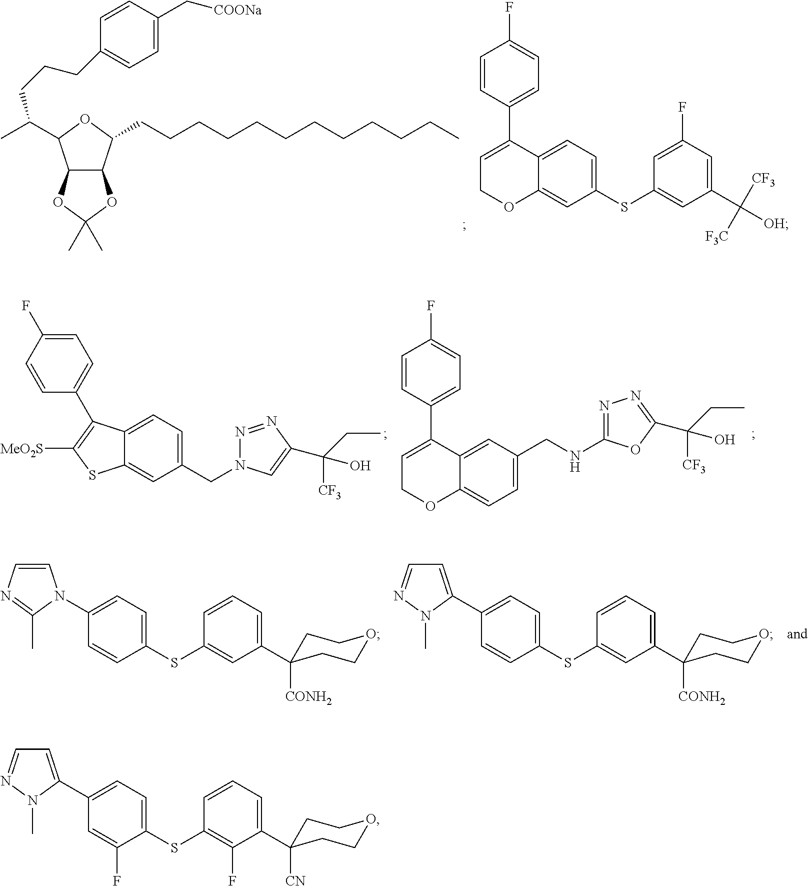
or a pharmaceutically acceptable salt thereof or stereoisomer thereof.

4. The method of claim 2, wherein the inhibitor of 5-lipoxygenase has the formula:

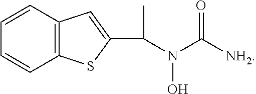 [SMILES: CC(c1cc2ccccc2s1)N(O)C(N)=O]

5. The method of claim 1, wherein the method further comprises administering at least one product of the cyclooxygenase pathways to the mammal.

6. The method of claim 5, wherein the at least one product of the cyclooxygenase pathways is prostaglandin E2.

7. (canceled)

8. The method of claim 1, wherein the inhibitor of the 5-lipoxygenase pathway is a leukotriene receptor antagonist or a lipoxin receptor antagonist.

9. The method of claim 8, wherein the inhibitor of the 5-lipoxygenase pathway is a leukotriene receptor antagonist selected from the group consisting of montelukast, zafirlukast, and pranlukast.

10. (canceled)

11. The method of claim 1, wherein the method further comprises administering at least one antimicrobial agent to the mammal.

12. The method of claim 11, wherein the antimicrobial agent is an antibiotic agent selected from the group consisting of isoniazid, rifampin, pyrazinamide, ethambutol, and combinations thereof.

13. (canceled)

14. The method of claim 11, wherein the antimicrobial agent is an antiprotozoal agent selected from the group consisting of melarsoprol, nifurtimox, pentamidine, sodium stibuglyconate, suramin, atovapuone, timidazole, dapsone, clofazinime, and rifampin, and combinations thereof.

15. (canceled)

16. The method of any claim 11, wherein the method results in enhancing the efficacy of the antimicrobial agent, wherein the enhancing results in reducing overall disease severity and mortality, reducing the length of antimicrobial treatment regimen, increased tolerance of the antimicrobial agent, or any combination thereof.

17. (canceled)

18. The method of claim 1, wherein the tuberculosis is selected from mycobacterium tuberculosis, a multi-drug resistant tuberculosis (MDR), and an extremely drug resistant tuberculosis (XRT).

19-24. (canceled)

25. A pharmaceutical composition comprising effective amounts of
(a) an inhibitor of the 5-lipoxygenase pathway, and/or
(b) at least one product of the cyclooxygenase pathways, and optionally
(c) an antimicrobial agent,

26. The composition of claim 25, wherein the at least one product of the cyclooxygenase pathways is prostaglandin E2.

27. The composition of claim 25, wherein the optional antimicrobial agent is an antibiotic or an antiprotozoal agent.

28. (canceled)

29. A kit for enhancing the effective immune response of a mammal in the treatment or prevention of a disease caused by intracellular microorganisms, wherein the kit comprises effective amounts of:
(a) an inhibitor of the 5-lipoxygenase pathway, and/or
(b) at least one product of the cyclooxygenase pathways, and instructions to treat or prevent a disease caused by intracellular microorganisms.

30. The kit of claim 29, wherein the at least one product of the cyclooxygenase pathways is prostaglandin E2.

31. The kit of claim 29, wherein the kit further comprises an antimicrobial agent, wherein the antimicrobial agent is an antibiotic or an antiprotozoal agent.

32-33. (canceled)

34. A method of treating or preventing a disease caused by intracellular microorganisms in a patient comprising administering to the patient effective amounts of:
(a) an antimicrobial agent and
(b) an inhibitor of the 5-lipoxygenase pathway, and optionally
(c) at least one product of the cyclooxygenase pathways.

35. The method of claim 34, wherein the at least one product of the cyclooxygenase pathways is prostaglandin E2.

36. The method of claim 34, wherein the antimicrobial agent is an antibiotic or an antiprotozoal agent.

37-39. (canceled)
